(19) United States
(12) Patent Application Publication
(10) Pub. No.: US 2009/0110717 A1
(43) Pub. Date: Apr. 30, 2009

(54) TRANSMUCOSAL COMPOSITION

(76) Inventors: Amarjit Singh, Mumbai (IN); Sarabjit Singh, Mumbai (IN); Shivanand Puthli, Mumbai (IN)

Correspondence Address: LADAS & PARRY LLP, 26 WEST 61ST STREET, NEW YORK, NY, 10023, US

(21) Appl. No.: 12/297,486

(22) PCT Filed: Apr. 25, 2007
(86) PCT No.: PCT/IN2007/000164

§ 371 Date: Oct. 17, 2008

(30) Foreign Application Priority Data
May 2, 2006 (IN) 682/MUM/2006

(51) Int. Cl. A61K 9/70; A61K 9/00; A61K 39/00; A61K 31/4178; A61K 31/4045; A61K 31/445; A61K 31/4375

(52) U.S. Cl. 424/448; 424/400; 424/184.1; 514/397; 514/415; 514/329; 514/283

(57) ABSTRACT

The invention provides a composition for delivering active agents through transmucosal administration, more particularly through the buccal mucosa. The composition is a unique transmucosal disk (10) which has two compartments (14) and (18); the compartments consist of at least one active agent and at least one mucoadhesive agent and both the compartments are adapted to be in contact with the mucosal membrane. The invention also provides for transmucosal administration of an active agent and method of treatment of diseases in a subject in need of such treatment.

### TECHNICAL FIELD

The present invention relates to a novel composition and method for delivering active agents through transmucosal administration, more particularly through the buccal mucosa of the oral cavity.

### BACKGROUND OF THE INVENTION

Transmucosal administration of biologically active agents through absorptive mucous membranes such as buccal, sublingual, ocular, nasal, pulmonary, rectal, and vaginal membranes has the advantage of being noninvasive and of bypassing hepato/gastrointestinal clearance. However, these mucosal membranes, depending on their functionality, have a specific cellular physiology, and in absence of external stimuli to facilitate absorption, have been found to exhibit limited permeability to most molecules. Novel compositions and methods are still in need to be developed for delivering active agents, especially the macromolecules and complex agents, through these membranes.

Among the various transmucosal routes, the buccal mucosa is easily accessible, has a wide area of smooth muscle and has relatively less mobility, making it suitable to administer retentive compositions. Absorption through the buccal mucosa delivers the active agents directly into the systemic circulation through the internal jugular vein, thereby bypassing the hepatic first pass metabolism. The mucosa also exhibits low enzymatic activity, avoids degradation in the gastric and intestinal fluids and is potentially more tolerant to permeation enhancers and pH modifiers as compared to other delicate membranes such as the nasal mucosa. The buccal mucosa is suitable for local as well as systemic delivery of active agents both as immediate delivery systems and as controlled or retentive delivery systems. Hence the intent of the inventors is to formulate a transmucosal composition which is effective and which exhibits satisfactory delivery of active agents, by using the buccal mucosa as a model transmucosal membrane.

In spite of the above mentioned advantages of the buccal mucosa, major limitations in the development of a novel composition are the barrier properties of the buccal cells, effect of salivary circulation and accidental swallowing of the composition.

Various dosage forms for buccal and other transmucosal administration of active agents are well known in the art. (Buccal bioadhesive drug delivery—A promising option for orally less efficient drugs; Sudhakar et al, J. Cont. Rel, 114, 15-40,2006) These commonly include films, patches, sprays, lozenges, gums, tablets etc.

The term “buccal patch” or “film” typically refers to a flexible film that adheres to the oral mucosa and delivers the active agent. Such films can be either quick dissolving or dispersing films releasing the active agent immediately or can be films having mucoadhesive properties with the active being released over a period of time. These patches or films are typically prepared by mixing the ingredients, heating, extruding, drying and then sizing the sheets to deliver the exact amounts of medications. (Polymeric Films As Vehicle For Buccal Delivery: Swelling, Mechanical, and Bioadhesive Properties; Wong et al, J. Pharm Pharmaceut Sci, 53-61, 1999) Such methods require relatively long time period for drying the films, need to maintain tight control over the drying conditions, and generally result in relatively high moisture content of the finished dosage form. Also, the heating step required may preclude the incorporation of heat-sensitive active agents.

Buccal sprays generally contain the actives along with solvents and optionally propellants. These sprays produce a fine mist of the active, which deposits onto the buccal mucosa, from where it is absorbed. (U.S. Pat. No. 5,955,098). The use of solvents, propellants can be toxic, expensive and requires special precautions during manufacture. Also, it is difficult to formulate prolonged release compositions by this method.

Lozenges and gums release the active as the dosage form is sucked, or chewed. (U.S. Pat. No. 5,549,906, U.S. Pat. No. 4,806,356). In addition to buccal absorption, most of the active agent released flows with the saliva into the gastric cavity. Also, the release shows variability depending on how fast it is chewed or sucked.

Buccal tablets are relatively convenient to manufacture and formulate. Matrix type tablets are prepared by dispersing the active agent in suitable bioadhesive polymers, and other excipients. Many employ the use of penetration enhancers for optimal penetration of the active agent through the mucosa. However, interactions between the active agent and the adhesives modify the drug release pattern, and may also cause instability of the system. One solution to this problem is the preparation of multilayered tablets, which segregate the interacting ingredients. However, multilayered tablets are often prone to the problem of separation of layers. Also, since only the adhesive layer is attached to the mucosa, the active agent in the opposing layer has to first enter this layer, and migrate through it to be available for absorption. Alternatively, the tablet has to be placed in the gingival cavity between the mucosa of the lips and the gum, so that the adhesive layer sticks to the gums and the drug is absorbed through the mucosa of the lips. See, for example U.S. Pat. No. 5,849,322. This type of dosage form faces restrictions on the location in the mouth where it can be placed.

U.S. Pat. No. 5,639,469 describes a transmucosal device for delivering a heparinic anticoagulant wherein the drug containing matrix reservoir is maintained in contact with the mucosal surface by an outer mucoadhesive portion disposed peripherally to the matrix. Such a device was prepared by solvent casting a sheet of mucoadhesive, die cutting into the sheet rings of specified diameters, and laminating a sheet of surgical dressing on one side. A matrix of gel, powder formulation or tablets was placed into the rings to form drug reservoirs. Such an elaborate method of preparation is generally labour intensive and not scale-up friendly.

All these above-mentioned limitations in the various buccal compositions have resulted in very few products that are viable commercially. There is a need for an improved composition for buccal administration of active agents which is efficient in all aspects i.e. which provides good stability and effectiveness, is versatile, easy to manufacture, user-friendly and allows for control over the dosage and effect of the active agents.

It is the object of the present invention to provide a novel composition and method for delivering an active agent by transmucosal administration, preferably through buccal mucosa.

An object of the present invention is to provide a novel composition for transmucosal administration of active agents, which is easy to manufacture, exhibits good stability and allows for flexibility of formulation.

Another object of the present invention is to provide a novel composition for transmucosal administration of active agents, which allows for precise control over the dose administered and the effect obtained.

Yet another object of the present invention is to provide a novel composition for transmucosal administration of active agents, which is simple, convenient to administer, easy to handle and promotes high patient acceptance and compliance.

### BRIEF DESCRIPTION OF THE INVENTION

It has been found that most of the above objects are realized by the composition and method of the invention. The composition is a unique transmucosal disk for administration of an active agent which contains two compartments; at least one active agent and at least one mucoadhesive agent are present in the compartments. The transmucosal disk is such that, on mucosal application, the compartments are in contact with one common mucosal membrane.

Preferably, the active agent and the mucoadhesive agent are present in separate compartments.

In an embodiment the transmucosal disk comprises of two compartments; an inner compartment which contains the active agent/s and an outer compartment which contains the mucoadhesive agent/s. The inner compartment is surrounded by the outer compartment around all its surfaces except one surface, so that on mucosal application this exposed surface comes in contact with the mucosal membrane and delivers the active agent, while the outer compartment contacts and adheres to the same mucosal membrane. This helps in direct absorption of the active agent from this compartment, while the surrounding mucoadhesive “ring” of the outer compartment adheres the disk in place.

The transmucosal disk of the present invention is prepared by the simple and commercially feasible technique of compression. The disk is robust, facilitating its handling and easy application. Also, the disk is easily removable and can be peeled off from the mucosal membrane when the desired effect of the active agent has been achieved.

The mucoadhesive agent used in the present invention can be a combination of fenugreek gum with polycarbophil in a ratio of about 1:5 to about 5:1. This combination is found to demonstrate optimal mucoadhesive properties, such that a composition has sufficient adhesion to be retained on a mucosal membrane intact for a desired period of time, while not so strong that there is abrasion of the mucosal membrane during peeling off. Hence the invention also provides for a composition for transmucosal administration of an active agent which contains a combination of fenugreek gum with polycarbophil in a ratio of about 1:5 to about 5:1.

In a preferred embodiment, the transmucosal disk of the present invention is applied to the buccal mucosa.

The present invention also provides for a method for transmucosal administration of an active agent wherein the transmucosal disk of the present invention is applied to a mucosal membrane and kept in contact with it for a therapeutically effective period of time. When the desired therapeutic effect has been achieved, the disk can be optionally peeled off.

The present invention further provides for a method of treatment and prophylaxis of diseases comprising administering to a subject in need of such treatment, the composition of the invention.

### DESCRIPTION OF THE DRAWINGS

The present invention will be described in additional specifics and detail through use of the accompanying drawings in which:

FIG. 1 depicts an illustrative transmucosal disk having the general configuration of the present invention.

FIG. 2 depicts the top view of differently shaped specific embodiments of the present invention.

FIG. 3 is a graph showing dissolution profile of desmopressin acetate composition of Example 3 in pH 6.8 buffer, USP Type II apparatus, over a period of 8 hours.

FIG. 4 is a graph showing the dissolution profile of ondansetron composition A of Example 4 in 0.1 N HCl, USP Type II apparatus. The composition demonstrates fast release, with almost complete dissolution within an hour.

FIG. 5 is a graph showing dissolution profile of sumatriptan succinate composition A of Example 5 in pH 6.8 buffer, USP Type II apparatus. The composition demonstrates fast release, with almost 80% of the active released in an hour.

### DETAILED DESCRIPTION OF THE INVENTION

For the purpose of the present invention, the term ‘active agent’ is defined as any substance, natural or synthetic, which induces a pharmacological or biological effect in a subject. Such substances are intended to furnish pharmacological activity or other direct effect in the diagnosis, cure, mitigation, treatment, or prevention of disease or to affect the structure and function of the body.

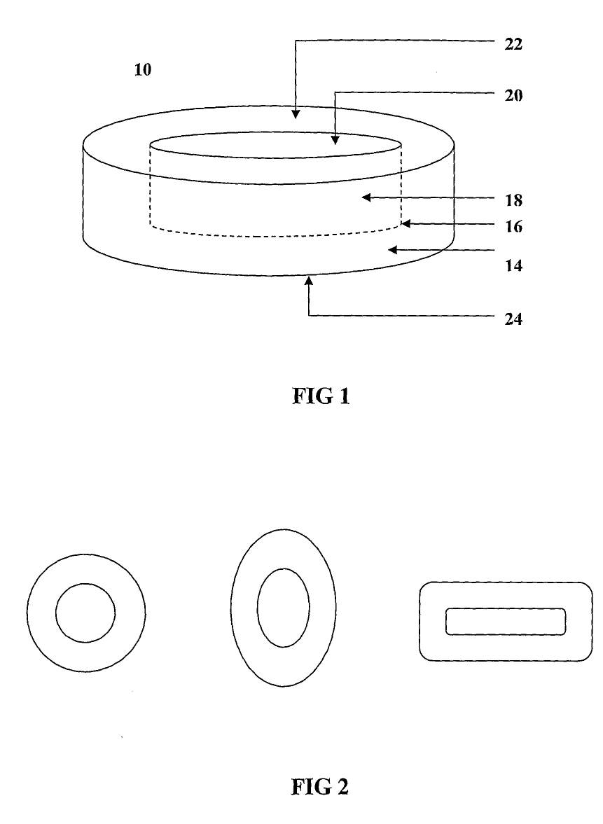

As disclosed in the present invention, the mucoadhesive ‘ring’ is the area of the outer compartment which surrounds the exposed surface of the inner compartment and which is in contact with and adheres to the mucosal membrane. (Surface 22 of the FIG. 1).

As disclosed in the present invention, the ‘transmucosal disk’ is the composition of the invention which is applied to a mucosal membrane and which is used to deliver an active agent through transmucosal administration. The composition can be of any shape and size as desired.

The following detailed description is not intended to limit the scope of the invention, as claimed, but is merely representative of the presently preferred embodiments of the invention.

Referring to FIG. 1 there is shown an illustrative transmucosal disk 10 according to the present invention for delivering an active agent through transmucosal administration, preferably through the buccal mucosa. In the figure the transmucosal disk 10 of a general overall configuration is shown, having an outer compartment 14 with a recess 16 in one side thereof. An inner compartment 18 fits into the recess such that one surface of the inner compartment is exposed, and the remaining surfaces are surrounded by the outer compartment. Both the compartments together form a unitary construction. The exposed surface 20 of the inner compartment is flush with the surrounding surface 22 of the outer compartment such that when the transmucosal disk adheres to a mucosal membrane, both the compartments are in contact with the mucosa.

The dimensions of both the compartments are chosen as per the active agent to be administered and its dosage. The ratio of lengths of both the compartments (i.e. diameters in case of a circular disk) is selected such that the surface 22 of the outer compartment has sufficient area to enable adequate adhesion of the disk to a mucosal membrane. Accordingly, a ratio between the length of the outer compartment and length of the inner compartment of more than 1.1:1, preferably more than 1.2:1, is preferred. The length of the disk preferably varies between 1 mm to 25 mm. Thickness of the disk is kept at a minimum possible, to ensure least possible foreign body sensation and better patient compliance and mouth-feel. Preferably, the thickness is about 0.5 mm to about 5 mm. The ratio of depths of both the compartments can be selected of any desired value, depending upon the needs of the specific formulation.

FIG. 2 depicts the top view of alternative shapes of the transmucosal disk which can be easily manufactured by compression techniques. The most preferred shape is the circular one, which requires least adjustments, with respect to orientation of both the compartments with each other, during manufacturing. However, other shapes such as oval, ellipsoidal, capsule shape, as shown in FIG. 2 are equally possible and are included in the scope of the invention. Such shapes lack sharp edges and are hence less prone to problems such as mechanical instability and uneven distribution of pressure during compression. However, it is to be understood that other modifications to the shape, such as making various geometric shapes or making both the compartments of different shapes are obvious to a person skilled in the art and are contemplated as part of the invention.

The inner compartment contains the active agent or agents and excipients. The active agent is present in the range of about 0.1 to about 99% w/w, preferably about 1 to about 90% w/w of the disc, depending on its dose and formulation factors. The active agents suitable for incorporation in the transmucosal disk are those which would show beneficial effects on mucosal administration. Such actives include those that require a fast onset of action, extended effects, are degraded in the gastric or intestinal juices, have an unpleasant taste, are degraded in the saliva, undergo pre-systemic clearance etc. In general, examples of categories of active agents which can be incorporated are anti-infectives such as antibiotics and antiviral agents; analgesics and analgesic combinations; anorexics; antihelminthics; antiarthritics; antiasthmatic agents; anticonvulsants; antidepressants; antidiabetic agents; antidiarrheals; antihistamines; antiinflammatory agents; antiinsomnia agents; antiemetics; antimigraine preparations; antinauseants; antineoplastics; antiparkinsonism drugs; antipruritics; antipsychotics; antipyretics; antispasmodics; anticholinergics; sympathomimetics; xanthine derivatives; cardiovascular preparations including potassium and calcium channel blockers, beta-blockers, alpha-blockers, and antiarrhythmics; antihypertensives; diuretics and antidiuretics; vasodilators including general coronary, peripheral and cerebral; central nervous system stimulants; vasoconstrictors; cough and cold preparations, including decongestants; hormones such as estradiol and other steroids, including corticosteroids; hypnotics; immunosuppressives; muscle relaxants; parasympatholytics; psychoanaleptics psychostimulants; sedatives; and tranquilizers; and agents for pain management of cancer. By the transmucosal disk of the present invention, both ionized and nonionized active agents may be delivered, as can be actives of either high or low molecular weight.

Of special relevance to the invention are active agents, which are proteins, peptides, polysaccharides, carbohydrates and other agents which exhibit extensive first pass metabolism. Proteins, peptides, carbohydrates are generally absorbed to a very less extent from the gastrointestinal tract, due to local degradation and first pass effect. Hence they are often given by parenteral routes, such as intravenously or subcutaneously. However, these routes have many disadvantages. Hence, the transmucosal disk of the invention would be particularly advantageous for such active agents. Examples of proteins and peptides that can be incorporated include, but are not limited to oxytocin, vasopressin, adrenocorticotrophic hormone, epidermal growth factor, prolactin, luliberin or luteinising hormone releasing hormone, growth hormone, growth hormone releasing factor, insulin, melatonin, somatostatin, glucagon, interferon, gastrin, tetragastrin, pentagastrin, urogastroine, secretin, calcitonin, enkephalins, endorphins, angiotensins, renin, bradykinin, bacitracins, polymixins, colistins, tyrocidin, gramicidines, and synthetic analogues, modifications and pharmacologically active fragments thereof, monoclonal antibodies, antigens and vaccines like those for anthrax, inluenza etc.

Examples of other active agents which undergo extensive first pass metabolism and which can be administered by the composition of the invention include agents like isosorbide mononitrate, isosorbide dinitrate, nifedipine, nitroglycerin, propranolol and lignocaine. The transmucosal disk of the invention can also be used to administer active agents having local effect like anesthetics, agents for treating mouth ulcers, toothaches, periodontal disease, bacterial and fungal infections etc.

Some examples of agents which can be administered include, without limitations, acyclovir, acetaminophen, amoxicillin, aripiprazole, aspirin, bupropion, buprenorphine, camptothecin analogs, celecoxib, cidofovir, clopidogrel, darifenacin, darunavir, desmopressin, diclofenac, duloxetine, eletriptan, emezine, enoxaparin, eszopiclone, etomidate, fentanyl, fondaparinux, gabapentin, granisetron, heparin, hormones and oral contraceptives, irbesartan, iron supplements, itraconazole, ketoprofen, levetiracetam, lidocaine, loperamide, ibandronic acid, meloxicam, mefenamic acid, memantine, metformin, metronidazole, miconazole, misoprostol, metaxolone, morphine, nebivilol, nicotine, olopatadine, ondansetron, oxcarbazepine, oxybutynin, oxycodone, oxymorphone, paliperidone, paracetamol, penciclovir, pilocarpine, pioglitazone, polidocanol, prochlorperazine, quetiapine, rosiglitazone, rivastigmine, sufentanyl, sumatriptan, tegaserod, thalidomide, tolterodine, tramadol, valdecoxib, zonisanide, zopiclone and zolpidem.

The inner compartment may also optionally include about 0.1 to about 90% w/w, preferably about 1 to about 80% w/w of absorption-promoting agents, also known as permeation enhancers, which increase the flux of the permeates across the mucosa. Membrane permeation is the limiting factor for many active agents in the development of transmucosal compositions. The epithelium that lines the buccal mucosa is a very effective barrier to the absorption of actives. Hence, agents that facilitate their permeation through buccal mucosa may be advantageously used in the transmucosal disk of the invention. Various absorption-promoting agents are known. For example chelators such as EDTA, citric acid, sodium salicylate; surfactants such as sodium lauryl sulphate, benzalkonium chloride, polyoxyethylene, 23-lauryl ether; bile salts such as sodium deoxycholate, sodium glycocholate, sodium taurocholate; fatty acids such as oleic acid, capric acid, lauric acid; non-surfactants such as cyclic ureas, cyclodextrins; and others such as polysorbates, aprotinin, azone, alkyl glycosides, chitosan, menthol, dextran sulfate etc. can be used. The selection of the permeation enhancer is done depending on factors such as physicochemical properties of the active agents, nature of excipients, toxicity and efficacy at buccal tissue etc. A combination of permeation enhancers can also be used if they exhibit a synergistic effect.

The transmucosal disc of the invention can be formulated for any type of release profile. This is achieved by the use of rate controlling polymers. The polymers are incorporated in the inner compartment by blending with the active agent/s or by coating over their particles. Depending upon the ingredients used, the active agent/s are released by processes of diffusion through or erosion of the inner compartment. The active agent thus released to the mucosa or in its vicinity is absorbed through the mucosal membrane from different pathways, depending upon the physicochemical properties of the active agent and presence of permeation enhancers.

The inner compartment may comprise of about 1 to about 99% w/w of rate controlling polymers. Examples of suitable rate controlling polymers which can be used include cellulosic polymers such as hydroxypropyl cellulose, hydroxyethyl cellulose, hydroxypropyl methyl cellulose, methyl cellulose, ethyl cellulose, cellulose acetate, cellulose acetate phthalate, cellulose acetate trimellitate, hydroxypropylmethyl cellulose phthalate, cellulose ester-ether phthalate, hydroxypropylcellulose phthalate, alkali salts of cellulose acetate phthalate, alkaline earth salts of cellulose acetate phthalate, hydroxypropylmethyl cellulose hexahydrophthalate, cellulose acetate hexahydrophthalate, and carboxymethylcellulose sodium; acrylic acid polymers and copolymers preferably formed from acrylic acid, methacrylic acid, acrylic acid alkyl esters, methacrylic acid alkyl esters, and the like, e.g. copolymers of acrylic acid, methacrylic acid, methyl acrylate, ethyl acrylate, methyl methacrylate and/or ethyl methacrylate, with a terpolymer of ethyl acrylate, methyl methacrylate and trimethylammonioethyl methacrylate chloride, polymers having dissociable carboxyl groups, other particularly preferred polymers like; vinyl polymers and copolymers such as polyvinyl pyrrolidone, polyvinyl acetate, polyvinylacetate phthalate, vinylacetate crotonic acid copolymer, and ethylene-vinyl acetate copolymers; alkylene oxide homopolymers such as polypropylene oxide, and ethylene oxide homopolymers; and shellac, ammoniated shellac, shellac-acetyl alcohol, and shellac n-butyl stearate.

The inner compartment also optionally includes other excipients, like diluents, fillers, binders, lubricants, glidants, flavoring agents, sweetening agents, coloring agents, stabilizers, enzyme inhibitors, lubricants and rate-controlling polymers.

The outer compartment surrounds the inner compartment around all but one surface and prevents loss of active agent to the surrounding. It contains mucoadhesive agent or agents in combination with suitable excipients. Mucoadhesive agent is present in the range of about 1% to about 99% w/w, preferably about 5 to about 95% w/w of the disc. A large, diverse group of materials have been known as mucoadhesives, ranging from substances of natural origin to biodegradable grafted copolymers. Typically, these adhesives are hydrophilic, e.g., water soluble or swellable materials, which attract water from biological surfaces. This water transfer leads to a strong adhesive interaction. When hydrated, these also tend to form viscous fluids which increase their retention on the biological surfaces. Categories of molecules utilized as mucoadhesives include natural and synthetic polymers, celluloses, acrylates, carbomers, natural gums, vinyl derivatives and combinations thereof. Examples of mucoadhesive agents are hydroxypropyl cellulose, hydroxypropyl methylcellulose, hydroxyethylcellulose, ethylcellulose, carboxymethyl cellulose, dextran, guar-gum, polyvinyl pyrrolidone, polyvinyl acetate, pectins, starches, gelatin, casein, acrylic acid polymers, polymers of acrylic acid esters, acrylic acid copolymers, vinyl polymers, vinyl copolymers, polymers of vinyl alcohols, carboxy vinyl polymers and copolymers, vinyl esters, alkoxy polymers, polyethylene oxide polymers, polyethers, and mixtures thereof. Any of such suitable mucoadhesive agents or their combinations can be utilized in the composition of the invention.

Preferred mucoadhesive agents are hydrophilic polymers and natural gums. Examples of preferred hydrophilic polymers are cellulosic polymers such as hydroxyethyl cellulose, hydroxypropyl cellulose, methyl cellulose, hydroxypropyl methylcellulose, hydroxyethyl methylcellulose, ethylhydroxyethyl cellulose, carboxymethyl cellulose and its salts and mixtures of two or more thereof; vinyl polymers such as polyvinyl acetate, polyvinyl pyrrolidone; and acrylic acid polymers and copolymers such as carbopol, polycarbophil etc. The transmucosal disc may contain about 5 to about 95% w/w of the hydrophilic polymers.

An embodiment contains about 25 to about 75% w/w of high viscosity hydroxypropyl methylcellulose polymer, preferably about 30 to about 70% w/w of hydroxypropyl methylcellulose (HPMC K4M) as the mucoadhesive agent.

Natural gums are polysaccharides of natural origin. Examples of some natural gums are carrageenan, konjac, sodium alginate, agarose, guar, pectin, tragacanth, acacia, arabic, dextran, gellan, xanthan, scleroglucan, hyaluronic acid, chitosan, fenugreek gum, locust bean gum etc. The transmucosal disc may consist of about 10 to about 80% w/w of such natural gums for mucoadhesive purposes. Also preferred are the combinations of natural gums with cellulosic, vinyl or acrylic acid polymers, such as a combination of a natural gum with a hydrophilic polymer in the ratio of about 1:10 to about 10:1.

It has been found out by the inventors that a combination of the natural gum—fenugreek gum with the acrylic acid polymer ‘polycarbophil’ (Noveon AA1®) in a ratio of about 1:5 to about 5:1 demonstrates optimal mucoadhesive properties. This combination renders to a transmucosal composition sufficient adhesion so that it is retained intact on a mucosal membrane for a desired period of time. At the same time, the mucoadhesion is not so strong that there is abrasion of the mucosal membrane when the composition is peeled off. Hence the invention also provides for a composition for transmucosal administration of an active agent which contains a combination of fenugreek gum with polycarbophil in a ratio of about 1:5 to about 5:1.

In a particularly preferred embodiment of the transmucosal disc of the invention, the outer compartment consists of a combination of fenugreek gum with polycarbophil (Noveon AA1®) in a ratio of about 1:5 to about 5:1.

In an embodiment, the disc consists of about 5 to about 40% w/w of fenugreek gum in combination with about 5 to about 40% w/w of polycarbophil (Noveon AA1®).

The outer compartment may also contain other excipients suitable for the composition. For example, it may optionally include flavors to mask any taste of the mucoadhesives, diluents to make up the bulk and lubricants to aid in compression.

The inner and the outer compartment may be of different color to distinguish the compartments and to facilitate proper application of the system by the user, wherein the inner compartment is directly placed in contact with the buccal mucosa.

The transmucosal disk of the invention has been found to be retained in the buccal cavity for periods longer than 3 hours, even up to 8 to 10 hours. The disk is capable of releasing the active agent during this period in a sustained manner. The release can be controlled to be slow enough to prevent losses due to saturation while being sufficiently fast to maintain concentration of the active agent in the therapeutic range.

The active agent is transmucosally administered to a subject by applying the disk to the mucosal membrane and keeping it in contact with the membrane for a therapeutically effective period of time. The disk adheres easily when applied with a slight pressure. Depending upon the active agent administered, the subject can be instructed to retain the disk for a specified period of time. The disk can be designed such that after the administration is complete, it will dissolve or disintegrate away, requiring no additional disposal. Optionally, when the desired therapeutic effect has been achieved, the disk can be peeled off.

Although especially useful for prolonged administration, the transmucosal disk of the invention can also be utilized for immediate administration of the active agents. In such cases, excipients such as disintegrants and dissolution aids are used to ensure quick delivery. Thus the disk can be designed for immediate release and for modified release which includes delayed, sustained, controlled, extended, pulsed or any other type of release of the active agent and also for a combination of immediate and modified release of active agent(s). Techniques for doing this are well known in the art.

The transmucosal disk of the present invention can easily be manufactured on an industrial scale and does not require the use of any special processes or equipments. It is manufactured by means of a compression machine assembly, capable of multiple compression steps. Such machine assemblies are generally available in two types: those where both the compartments are compressed on a single machine and those where the inner compartment is compressed on one machine, and then transferred to another machine, where the outer compartment gets compressed over it.

In practice, the inner compartment is compressed in a similar fashion as a normal tablet. Almost any formula which will produce a firm tablet is satisfactory for the machines described. Blend of the active agent and excipients, comprising preformed granules or directly compressible excipients, is compressed on the first press to produce the inner compartment. Weight and hardness are adjusted so that there is sufficient mechanical stability to withstand the transfer to the next compression stage. The transfer can be automatic through a transferring arm and cup. Alternatively, in some machines, the compressed inner compartments are filled into a vibrating feeder or a hopper, from which they are fed onto feeding discs or flexible feeder tubes and deposited into the second compression die cavity. The compressed inner compartment is positioned in the centre of the second compression die cavity. The blend of the outer compartment is fed into the die and then compressed around the sides and the top surface of the inner compartment.

The outer compartment composition requires good adhesiveness to the inner compartment and cohesion to make the disk physically stable. It should also exhibit some degree of plasticity to accommodate the expansion of the inner compartment after ejection from the die. The mucoadhesives used in the current invention, generally satisfy these requirements and lead to formation of a stable and robust transmucosal disk.

Compositions with similar general construction have earlier been discussed in the prior art. See, for example, U.S. Pat. No. 3,048,526; U.S. Pat. No. 6,251,848. They have been evaluated for uses such as for making detergents, for oral administration of a molecule with two delivery rates, or for administration of two agents. Such compositions were prone to problems. The manufacturing parameters had to be set carefully, otherwise problems such as tilting and off-centering of cores, inadequate adhesion of layers, and wastage due to many rejects were common. With the advent of film coating and other improved technologies for drug delivery, use of these compositions fell into disfavor. Film coating and better technologies for multiple drug delivery systems are both cost-effective and simple for manufacturing. As a result, such constructions are rarely, if ever, investigated and used now.

The inventors of the present invention have developed improved specific compositions using such general construction, which not only solve most of the problems of the prior art, but are also particularly useful for the purpose of the invention, i.e. to provide novel and effective composition and method for transmucosal administration of active agents.

The advantage of the transmucosal disk of the present invention is that it can be placed in a body cavity, such as the buccal cavity, such that the exposed surface of the inner compartment is in direct contact with the mucosal membrane. This helps in direct absorption of the active agent from its compartment, while the surrounding mucoadhesive “ring” of the outer compartment adheres the disk in place. A subject can peel off the disk when the desired effect has been achieved. This practice is called ‘dose-to-effect’, where the subject can regulate the administration of dose till the desired therapeutic effect has been achieved. In conditions such as pain, migraine, nausea, motion sickness etc. each subject needs different amounts of medication to treat his symptoms and he can decide the dose to be administered. Moreover, it has been well documented that there are wide variations in drug disposition characteristics between people of different demographic origins and races. Thus the amount of active agent required to produce a therapeutic end-point also differ. In such cases, doses can be easily titrated on an individual basis, so that once the appropriate amount of active agent has been administered, the subject can remove the disk, thereby stopping further absorption and overdose. Thus the composition of the invention allows precise control over the dose administered and the effect obtained.

Alternatively, the disk can also be designed such that after the administration is complete, the compartments will dissolve or disintegrate away, requiring no additional disposal.

Most of the retentive transmucosal compositions, especially those to be placed in the buccal cavity, face the problem of being such that they either get detached from the site of adhesion before the active agent is delivered due to inappropriate adhesion or they adhere so strongly that there is abrasion and peeling-off of the epithelial region while trying to remove the composition from the place. The transmucosal disk of the invention has exhibited sufficient adhesion for many hours and at the same time is easily peel able. Similarly, being a compressed, robust disk, unlike other buccal films or patches which are generally fragile, it is convenient to handle and easy to administer. All these characteristics promote high patient acceptance and compliance.

Presence of active agents in the inner compartment allows even unpleasant tasting actives to be administered. This is a huge advantage considering that unpleasant taste of many active agents precludes their administration by the oral transmucosal route. Also, another category of active agents which can be beneficially administered by the transmucosal disk of the present invention are those which are degraded in the salivary fluid. Such actives are generally unstable in the presence of salivary pH and enzymes such as amylases. Inclusion of such agents in the inner compartment ensures minimal contact with the saliva and hence improved stability.

The transmucosal disk is simple and other than a specialized compression step, does not involve any complex process or equipment. The robustness of the compressed tablet form makes packaging and transporting also convenient. Segregation of active agents and mucoadhesive agents in separate compartments minimizes their interaction and improves stability. Also, presence of the active agent in the inner compartment ensures that it is unidirectional absorbed directly through the mucosal membrane and is prevented from getting swallowed with saliva.

The transmucosal disk is also flexible in that it can be easily adapted for any kind of delivery profile. For example it can be used for immediate release, sustained release, pulsatile release, delayed release and any other type of controlled release.

Buccal cavity is the part of mouth bounded on one side by the teeth and gingivae (or the residual alveolar ridges), and on the other by the cheeks. One advantage of the transmucosal disk of the present invention is that it can be placed virtually anywhere in the buccal cavity, either in the gingival compartment or on either of the cheek mucosa.

The other advantages of the present invention are reduced chances of dose dumping, unnecessary burst effects and failure of the system, which are otherwise usually associated with simple matrix or reservoir systems.

### In-Vivo Mucoadhesion Trial:

Illustrative transmucosal disk of the invention was evaluated for its mucoadhesive properties and suitability as buccal composition. 6 healthy volunteers were selected for in vivo determination of properties such as adhesion, residence time, swelling capacity, mouth feel etc. Two compositions coded L and R were evaluated in each volunteer. General method of manufacturing described for Examples 1 to 7 was used to prepare the compositions. Composition L was prepared using a combination of Polycarbophil and Fenugreek Gum as the mucoadhesive agent. Composition R contained a combination of Polycarbophil, Fenugreek Gum and Hydroxypropyl methyl cellulose (HPMC). The inner compartments were formulated as placebos containing diluents microcrystalline cellulose, Lactose, and Magnesium stearate. After intake of meals, a transmucosal disk was placed on the cheek of each volunteer with the inner compartment facing the cheek and then applying pressure, thereby adhering it in place. After 8 hours, the study period was terminated by rinsing the mouth with cold water and removing the disk. Observations of volunteers were recorded for various properties and are presented below:

TABLE 1

| | Property evaluated | | | | | |
|---|---|---|---|---|---|---|
| | | | Residence | | | |
| | Adhesion to | | time | | | |
| | mucosa | | (hrs) | | Water rinse test | |
| | Composition | | | | | |
| Volunteer | L | R | L | R | L | R |
| A | OK | OK | 7 | 7 | — | Removed by 4-5 |
| | | | | | | rinses. |
| B | Good | Good | 7.5 | 7.5 | Not removable by | Not removable by |
| | | | | | rinsing. | rinsing. |
| C | Good | Good | 7.5 | 7.5 | Not removed till 30 | Not removable by |
| | | | | | rinses, tablet | rinsing. |
| | | | | | disintegrated. | |
| D | Adhered | Adhered | 7 | 8 | Not removable by | Not removable by |
| | by | by | | | rinsing. | rinsing. |
| | applying | applying | | | | |
| | pressure | pressure | | | | |
| E | Good | Good | 8 | 7 | Removed by 2 | Not removable by |
| | | | | | rinses. | rinsing. |
| F | Good | Good | 7 | 7 | Dissolved by | Partially detached on |
| | | | | | rinsing. | several rinses. |

| | Property evaluated | | | | | |
|---|---|---|---|---|---|---|
| | Swelling Capacity | | Mouth feel | | Taste factor | |
| | Composition | | | | | |
| Volunteer | L | R | L | R | L | R |
| A | Slight | Slight | Minimal | Minimal | Slightly bitter | Slightly bitter |
| B | Minimal | Minimal | Slight metallic | None | Slight metallic | Tasteless |
| | | | taste | | taste | |
| C | None | None | Slight metallic | Minimal | Slight metallic | Almost |
| | | | taste | | taste | tasteless |
| D | Minimal | More than L | Dry mouth | Dry mouth | Tasteless | Tasteless |
| E | Minimal | OK | Minimal, with | Minimal, | No specific | No specific |
| | | | some saliva | with some | taste | taste |
| | | | secretion | saliva | | |
| | | | | secretion | | |
| F | Partial | Minimal | Minimal | Minimal | Tasteless | Tasteless |

The results indicate satisfactory mucoadhesive characteristics demonstrated by the transmucosal disk. The adhesion was good and effective for the entire period of the study and there was only minimal foreign body sensation and taste. The disks were not easily detached by rinsing and showed minimal swelling. Also, both the disks were found to be comparable.

Having described the invention, modifications to it will be apparent to a person skilled in the art and are included in the spirit scope of the invention. For example an impermeable backing layer may be formulated on the bottom side (Surface 24 of FIG. 1) of the disk. Similarly, the disk may be modified to include the active agent in the outer or both the compartments and the mucoadhesive agent in the inner or both compartments. The disk may also be adapted to deliver two agents, one in each compartment. Also, although the transmucosal disk has been described with reference to buccal administration, it is to be understood that it can be adapted and used for any suitable type of transmucosal administration.

The following non-limiting examples are illustrative of the embodiments of the invention and shall not be construed to limit the scope of the invention.

### EXAMPLE 1

| | |
|---|---|
| Ingredient | mg/tab |

Inner Compartment

| | |
|---|---|
| Desmopressin acetate | 0.1 |
| Lactose monohydrate | 49.4 |
| Magnesium stearate | 0.5 |

Outer Compartment

| | |
|---|---|
| Carbopol 974P | 40 |
| Microcrystalline cellulose | 98.6 |
| Magnesium stearate | 1.4 |

Components of the inner compartment were blended thoroughly and lubricated by means of magnesium stearate in a suitable blender. The blend was compressed on a rotary tablet compression machine using flat punch tooling, to form the inner compartment. Similarly, the contents of the outer compartment were blended and lubricated. The inner compartment was transferred to the centre of the second compression die cavity. The blend of the outer compartment was fed into the die and compressed around the inner compartment.

### EXAMPLE 2

| | |
|---|---|
| Ingredient | mg/tab |

Inner Compartment

| | |
|---|---|
| Desmopressin acetate | 0.1 |
| Lactose monohydrate | 15.6 |
| Hydroxypropyl methylcellulose | 15.0 |
| Magnesium stearate | 0.5 |

Outer Compartment

| | |
|---|---|
| Polyvinyl acetate | 40 |
| Microcrystalline cellulose | 98.6 |
| Magnesium stearate | 1.4 |

Manufacturing process is same as described in Example 1 above.

### EXAMPLE 3

| | |
|---|---|
| Ingredient | mg/tab |

Inner Compartment

| | |
|---|---|
| Desmopressin acetate | 0.10 |
| Lactose monohydrate | 31.20 |
| Microcrystalline cellulose | 6.00 |
| Magnesium stearate | 0.80 |

Outer Compartment

| | |
|---|---|
| Hydroxypropyl methyl cellulose | 90.00 |
| Microcrystalline cellulose | 59.25 |
| Magnesium stearate | 0.75 |

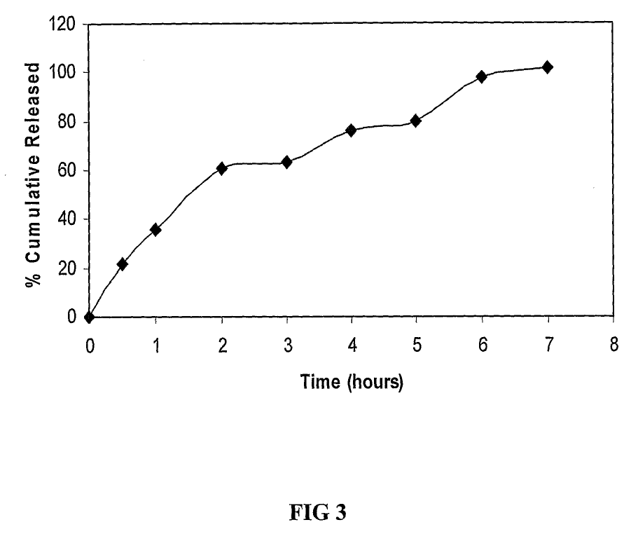

Manufacturing process is same as described in example 1 above. The disk was subjected to dissolution studies in pH 6.8 buffer, USP Type II apparatus. The dissolution profile achieved, as shown in FIG. 3 demonstrates sustained release over a period of 8 hours.

### EXAMPLE 4

| | mg/tab | | | | |
|---|---|---|---|---|---|
| Ingredients | A | B | C | D | E |
| Inner Compartment | | | | | |
| Ondansetron HCl | 6 | 6 | — | — | — |
| Ondansetron Base | — | — | 4.8 | 4.8 | 4.8 |
| Microcrystalline | 6 | 6 | 6 | 6 | 6 |
| cellulose | | | | | |
| Lactose monohydrate | 25.40 | 25.4 | 26.6 | 26.5 | 26.6 |
| Sodium Carbonate | — | 10 | — | — | — |
| Citric Acid | — | — | — | — | 10 |
| Sodium Taurocholate | — | — | — | 0.1 | — |
| Sodium Starch Glycolate | 2 | 2 | 2 | 2 | 2 |
| Magnesium Stearate | 0.6 | 0.6 | 0.6 | 0.6 | 0.6 |
| Outer Compartment | | | | | |
| Polycarbophil | 22.5 | 22.5 | 22.5 | 22.5 | 22.5 |
| Fenugreek Gum | 22.5 | 22.5 | 22.5 | 22.5 | 22.5 |
| Hydroxypropyl methyl | 104.25 | 104.25 | 104.25 | 104.25 | 104.25 |
| cellulose | | | | | |
| Magnesium Stearate | 0.75 | 0.75 | 0.75 | 0.75 | 0.75 |

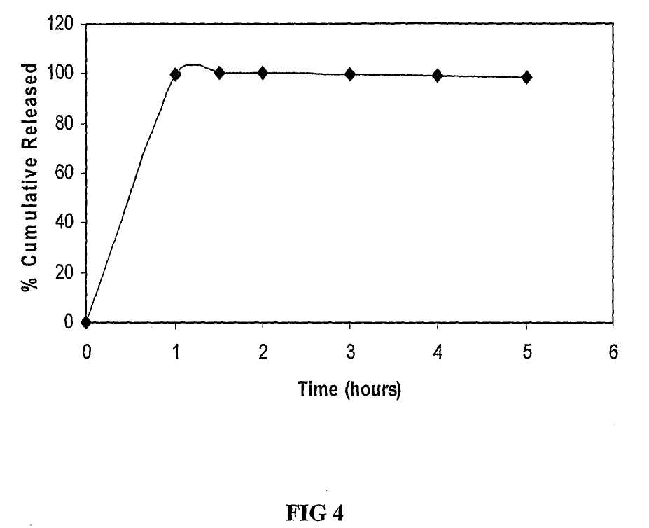

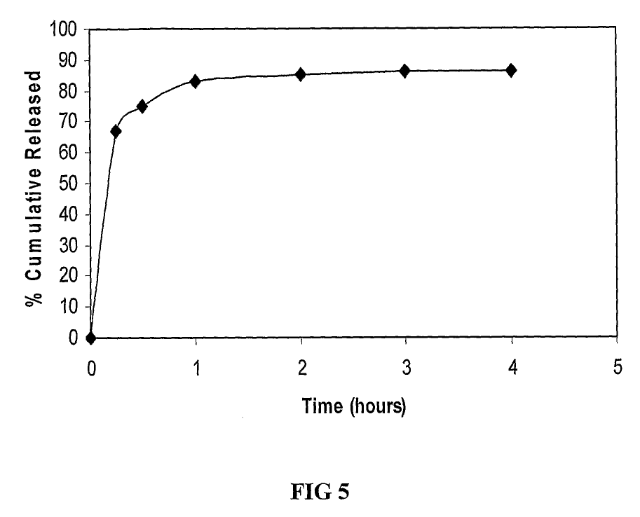

Above Table shows six different ondansetron compositions of the invention. Manufacturing process utilized was same as in Example 1. An illustrative composition A was subjected to dissolution studies in 0.1N HCl, in USP Type II apparatus. As shown in FIG. 4, the composition demonstrates fast release, with almost complete dissolution within one hour.

### Ex Vivo Permeation Study Using Porcine Buccal Mucosa:

To investigate transmucosal absorption of active agent from the composition of the invention, permeation study was carried out in Franz diffusion cell using excised porcine buccal mucosa. In the cell, the buccal membrane was equilibrated with Krebs buffer (pH 6.6). Composition A of Ondansetron described above was placed in the donor chamber and adhered to the moist membrane. Aliquots of buffer were withdrawn from the receiver chamber at regular intervals and assayed for the amount of ondansetron permeated through the membrane. From the results, the flux value was calculated. Flux is the slope of the amt of active permeated per unit area (Q/A) V/s time (t). It is a measure of the amount of active agent permeated through the membrane. Flux for the Composition A was calculated to be: 15.783 ug/h/cm^{2,} indicating significant permeation of Ondansetron from the composition of the invention.

### EXAMPLE 5

mg/tab

| Ingredient | A | B |
|---|---|---|
| Inner comportment | | |
| Sumatriptan Succinate | 35 | 35 |
| Hydroxypropyl methyl Cellulose | 6 | 6 |
| Lactose | 6.4 | 6.4 |
| Sodium carbonate | — | 10 |
| Sodium Starch Glycolate | 2 | 2 |
| Magnesium Stearate | 0.6 | 0.6 |
| Outer compartment | | |
| Hydroxypropyl methyl cellulose | 80 | 80 |
| Microcrystalline cellulose | 69.25 | 69.25 |
| Magnesium Stearate | 0.75 | 0.75 |

Two exemplary compositions of Sumatriptan are given in the table. Manufacturing process utilized was same as in Example 1. Composition A was subjected to dissolution studies in pH 6.8 buffer, in USP Type II apparatus. As shown in FIG. 5, the composition demonstrates fast release, with almost 80% release in an hour.

### Ex Vivo Permeation Study Using Porcine Buccal Mucosa:

Permeation study for Sumatriptan was also carried out, procedure being same as described in Example 4. The flux value calculated was found to be 22.084 ug/h/cm², indicating significant permeation of Sumatriptan from the composition of the invention.

### EXAMPLE 6

| | |
|---|---|
| Ingredient | mg/tab |

Inner Compartment

| | |
|---|---|
| Fentanyl citrate | 100.0 |
| Mannitol | 20.0 |
| Sodium starch glycolate | 7.5 |
| Magnesium stearate | 1.5 |

Outer Compartment

| | |
|---|---|
| Polycarbophil | 37.35 |
| Fenugreek gum | 37.35 |
| Microcrystalline cellulose | 174.05 |
| Magnesium stearate | 1.25 |

A fentanyl composition was made as per the invention. Manufacturing process followed was the same as Example 1.

### EXAMPLE 7

| | |
|---|---|
| Ingredient | mg/tab |

Inner Compartment

| | |
|---|---|
| Irinotecan hydrochloride | 5.0 |
| trihydrate | |
| Microcrystalline cellulose | 6.0 |
| Lactose monohydrate | 24.34 |
| Sodium starch glycolate | 2.0 |
| Magnesium stearate | 0.6 |

Outer Compartment

| | |
|---|---|
| Polycarbophil | 22.5 |
| Xanthan gum | 22.5 |
| Microcrystalline cellulose | 104.25 |
| Magnesium stearate | 0.75 |

An irinotecan composition was made as per the invention. Manufacturing process followed was the same as Example 1.

The composition of the invention can also be used to deliver a combination of active agents. For example, camptothecin derivatives like irinotecan can be given in combination with agents such as loperamide, anti-emetics like ondansetron, prochlorperazine etc. In an embodiment, the inner compartment contains a combination of irinotecan with loperamide immediate release granules. The granules of loperamide dissolve away, creating channels for the delivery of irinotecan.

1. A transmucosal disk for administration of an active agent comprising two compartments, wherein the compartments comprise of at least one active agent and at least one mucoadhesive agent and wherein the compartments are adapted to be in contact with one common mucosal membrane.

2. The transmucosal disk of claim 1 wherein the active agent and the mucoadhesive agent are present in separate compartments.

3. The transmucosal disk of claim 1 comprising, an inner compartment comprising at least one active agent and an outer compartment comprising at least one mucoadhesive agent, wherein the inner compartment is surrounded by the outer compartment around all its surfaces except one surface, said surface being adapted to be in contact with and deliver the active agent to a mucosal membrane when the outer compartment is in contact with and adheres to the same mucosal membrane.

4. The transmucosal disk of claim 1, wherein the transmucosal disk is prepared by compression.

5. The transmucosal disk of claim 1, wherein the transmucosal disk is adapted for easy application and removal from the mucosal membrane.

6. The transmucosal disk of claim 1, wherein the mucosal membrane is the buccal mucosa.

7. The transmucosal disk of claim 1, wherein active agent is selected from the group of anti-infectives, antibiotics, antivirals, analgesics, anorexics, antigens, antihelminthics, antiarthritics, antiasthmatic agents, anticonvulsants, antidepressants, antidiabetics, antidiarrheals, antihistamines, antiinflammatory agents, antiinsomnia agents, antiemetics, antimigraine agents, antinauseants, antineoplastics, antiparkinsonism agents, antipruritics, antipsychotics, antipyretics, antispasmodics, anticholinergics, sympathomimetics, xanthine derivatives, potassium and calcium channel blockers, beta-blockers, alpha-blockers, antiarrhythmics, antihypertensives, diuretics, antidiuretics, vasodilators, central nervous system stimulants, vasoconstrictors, cough and cold preparations, decongestants, hormones, hypnotics, immunosuppressives, muscle relaxants, proteins, peptides, polysaccharides, carbohydrates, parasympatholytics, psychoanaleptics, psychostimulants, sedatives, tranquilizers, vaccines and agents for pain management of cancer.

8. The transmucosal disk of claim 1, wherein active agent is selected from the group of anesthetics, agents for treating mouth ulcers, agents for treating toothaches, agents for treating periodontal disease and agents for treating bacterial and fungal infections.

9. The transmucosal disk of claim 1, wherein active agent is selected from the group of acyclovir, acetaminophen, amoxicillin, aripiprazole, aspirin, bupropion, buprenorphine, camptothecin analogs, celecoxib, cidofovir, clopidogrel, darifenacin, darunavir, desmopressin, diclofenac, duloxetine, eletriptan, emezine, enoxaparin, eszopiclone, etomidate, fentanyl, fondaparinux, gabapentin, granisetron, heparin, hormones and oral contraceptives, irbesartan, iron supplements, itraconazole, ketoprofen, levetiracetam, lidocaine, loperamide, ibandronic acid, meloxicam, mefenamic acid, memantine, metformin, metronidazole, miconazole, misoprostol, metaxolone, morphine, nebivilol, nicotine, olopatadine, ondansetron, oxcarbazepine, oxybutynin, oxycodone, oxymorphone, paliperidone, paracetamol, penciclovir, pilocarpine, pioglitazone, polidocanol, prochlorperazine, quetiapine, rosiglitazone, rivastigmine, sufentanyl, sumatriptan, tegaserod, thalidomide, tolterodine, tramadol, valdecoxib, zonisanide, zopiclone and zolpidem.

10. The transmucosal disk of claim 1, wherein the mucoadhesive agent is a hydrophilic polymer selected from the group of cellulosic polymers and copolymers, vinyl polymers and copolymers and acrylic acid polymers and copolymers.

11. The transmucosal disk of claim 1, wherein the mucoadhesive agent is a natural gum.

12. The transmucosal disk of claim 1, wherein the mucoadhesive agent is a combination of a natural gum and a hydrophilic polymer in a ratio of about 1:10 to about 10:1.

13. The transmucosal disk of claim 12, wherein the mucoadhesive agent is a combination of fenugreek gum and polycarbophil in a ratio of about 1:5 to about 5:1.

14. The transmucosal disk of claim 1, wherein the disk provides immediate release of active agent.

15. The transmucosal disk of claim 1, wherein the disk provides modified release of active agent.

16. A composition for transmucosal administration of an active agent comprising a combination of fenugreek gum with polycarbophil in a ratio of about 1:5 to about 5:1.

17. A method for transmucosal administration of an active agent comprising the steps of: providing a transmucosal disk comprising two compartments, wherein the compartments comprise of at least one active agent and at least one mucoadhesive agent and wherein the compartments are adapted to be in contact with one common mucosal membrane; applying the transmucosal disk to the mucosal membrane of a subject;
keeping the transmucosal disk in contact with the mucosal membrane for a therapeutically effective period of time; and optionally
peeling off the transmucosal disk when a desired therapeutic effect has been achieved.

18. The method according to claim 17, wherein the active agent and the mucoadhesive agent are present in separate compartments.

19. The method according to claim 17 wherein the disk is prepared by compression.

20. The method according to claim 17, wherein the disk is adapted for easy application and removal from the mucosal membrane.

21. The method according to claim 17, wherein the mucosal membrane is the buccal mucosa.

22. A method of treatment and prophylaxis of a disease comprising administering to a subject in need of such treatment the transmucosal disk of claim 1.
